(19) United States
(12) Patent Application Publication
(10) Pub. No.: US 2013/0130965 A1
(43) Pub. Date: May 23, 2013

(54) METHOD TO DETECT AND TREAT INFECTIOUS OR INFLAMMATORY DIARRHEA BASED ON REG1

(71) Applicants: William A. Petri, JR., Charlottesville, VA (US); Kristine Peterson, Charlottesville, VA (US)

(72) Inventors: William A. Petri, JR., Charlottesville, VA (US); Kristine Peterson, Charlottesville, VA (US)

(73) Assignee: University of Virginia Patent Foundation, Charlottesville, VA (US)

(21) Appl. No.: 13/639,993

(22) PCT Filed: Apr. 8, 2011
(86) PCT No.: PCT/US11/31680

§ 371 Date: Jan. 28, 2013

Related U.S. Application Data
(60) Provisional application No. 61/322,088, filed on Apr. 8, 2010.
(60) Provisional application No. 61/423,188, filed on Dec. 15, 2010.

(51) Int. Cl. A61K 38/17; G01N 33/68; G01N 33/569; A61K 39/02; C12Q 1/68

(52) U.S. Cl. CPC G01N 33/68
USPC 514/1.1; 435/7.92; 506/9; 435/6.12

(57) ABSTRACT

The present invention relates to methods for the diagnosis, treatment and prevention of infectious and/or inflammatory diarrhea.

### RELATED APPLICATIONS/PATENTS

The application claims the benefit of priority to U.S. Provisional Patent Application Ser. No. 61/322,088, filed Apr. 8, 2010, and U.S. Provisional Patent Application Ser. No. 61/423,188, filed Dec. 15, 2010, which applications are hereby incorporated by reference in their entirety.

### STATEMENT OF GOVERNMENT RIGHTS

This invention was made with government support under Grant No. R01 A1026649-19 awarded by the National Institutes of Health. The government has certain rights in the invention.

### BACKGROUND OF THE INVENTION

Diarrhea, also spelled diarrhea, is defined by the World Health Organization as having 3 or more loose or liquid stools per day, or as having more stools than is normal for that person (“Diarrhea”. World Health Organization). It is a common cause of death in developing countries and the second most common cause of infant deaths worldwide. The loss of fluids through diarrhea can cause dehydration and electrolyte imbalances. In 2009 diarrhea was estimated to have caused 1.1 million deaths in people aged 5 and over and 1.5 million deaths in children under the age of 5 (“The Basics of Diarrhea” Webmd.com).

Regenerating protein 1 (Reg1) is produced by the pancreas as well as by gastric, small bowel and colonic intestinal epithelial cells. Reg1A and 1B encode distinct proteins of 166 amino acids that are 87% identical and encoded by genes that are tandemly arrayed on chromosome 2p12. The Reg family of proteins includes 4 subclasses of which Reg1; A & B are in type I (1-3).

Reg1 expression is increased during intestinal inflammation (4-8). It is also expressed at high levels in colorectal, liver and bladder cancer (9-11). Reg1 expression is induced by gastrin, IL-6 and IFN-γ and STAT3. Functionally, Reg1 is anti-apoptotic and pro-proliferative for pancreatic islets and intestinal epithelium, an effect mediated in part via Bcl-xL, Akt and Bad (11-12). Reg1 is secreted and the Reg1 receptor is present on intestinal epithelial cells (13).

### SUMMARY OF THE INVENTION

The present invention is based on the discovery described herein that Reg1 expression is induced in colonic epithelium in patients with colitis due to amebiasis or C. difficile, that mice genetically deficient in Reg1 have increased susceptibility to and severity of infectious and/or inflammatory diarrhea, including diarrhea caused by Clostridium difficile, and that Reg1 can be detected in the stool of patients with intestinal inflammation, infection and diarrhea.

One embodiment of the invention provides compositions and methods for the diagnosis of infectious and/or inflammatory diarrhea, including diarrhea caused by Clostridium difficile. One embodiment provides for methods for the diagnosis and/or treatment of infectious, inflammatory diarrhea.

One embodiment provides for diagnosis of infectious and/or inflammatory diarrhea by the identification of Reg1 protein in the stool. One embodiment provides a method for diagnosing infectious or inflammatory diarrhea comprising detecting the presence of Reg1 protein or mRNA in a fecal sample or intestinal biopsy from a subject, wherein the presence of Reg1 protein or mRNA in the fecal sample or intestinal biopsy indicates that the subject has infectious and/or inflammatory diarrhea (as compared to a healthy individual (e.g., an individual without diarrhea). Another embodiment provides compositions, methods and kits for detecting Reg1 proteins or mRNA in the stool or intestinal biopsies.

Another embodiment provides a method for determining if treatment of infectious or inflammatory diarrhea is efficacious comprising detecting the presence of Reg1 protein or mRNA in a fecal sample or intestinal biopsy from a subject, wherein the presence of a reduced amount Reg1 protein or mRNA in the fecal sample or intestinal biopsy as compared to a sample from the subject taken earlier indicates that the treatment is effective, while in contrast a rising level (or static level) can indicate that the patient is relapsing with his/her infection or inflammation of the intestine and/or that therapy is ineffective.

In one embodiment, Reg1 protein is detected by means of immunological methods including contacting the sample with at least one reagent containing at least one antibody which binds to a Reg1 protein. In one embodiment, the antibody is detected using at least one of enzyme-linked immunoassay, western blot, lateral flow membrane test, latex agglutination, other forms of immunochromatography or immunoassay utilizing at least one antibody. In one embodiment, Reg1 proteins are detected using an ELISA. In one embodiment, Reg1 mRNA is detected using RT-PCR.

In one aspect, the Reg1 protein is Reg1A. In one aspect, the Reg1 protein is Reg1B. One embodiment provides for the use of both Reg1A and Reg1B. Another embodiment provides for the detection of both Reg1A and Reg1B. One embodiment provides for the detection of lactoferrin in combination with detecting Reg1A and/or Reg1B protein.

In one embodiment, the subject is human. In another embodiment, the diarrhea is caused by a bacterium or parasite. In one embodiment, the bacterium or parasite is a Campylobacter jejuni, Salmonella species, Shigella species, Escherichia coli (including enterohemorrhagic, enterotoxigenic, enteroaggregative E. coli), Entamoeba histolytica, Clostridium difficile, Cryptosporidium or a combination thereof.

In one embodiment, the diarrhea is caused by Clostridium difficile. In another embodiment, the diarrhea is caused by Entamoeba histolytica. In another embodiment, the diarrhea is caused by inflammatory bowel disease, Crohn's Disease (CD), ulcerative colitis (UC), or other chronic noninfectious inflammatory diarrheas.

One embodiment comprises informing a health care provider of the results of the assay/diagnosis and/or that the person should be treated. Another embodiment provides that the subject is treated for diarrhea or its causative agent following diagnosis of infectious or inflammatory diarrhea. One embodiment provides for the monitoring of amounts of Reg1 proteins during the treatment of the subject so as to determine if the treatment is effective.

Another embodiment provides for the method to be computerized and/or comprises the outputting of results to at least one of a display or a memory.

The availability of reagents to detect expression of Reg1 (protein or mRNA) makes application of the invention feasible and immediate.

The present invention provides advantages over current technology. For example, there are few intestinal biomarkers to detect or follow the course of intestinal infection or predict relapse, in particular, biomarkers that can be detected by a noninvasive method, such as by fecal samples.

The present invention also provides compositions and methods for the prevention and/or treatment of infectious and/or inflammatory diarrhea, including but not limited to, C. difficile infection.

In one embodiment, the present invention provides for the prevention or prophylactic treatment of infectious and/or inflammatory diarrhea by administrating Reg1 protein, or an active fragment or homolog thereof, or an isolated nucleic acid comprising a sequence encoding Reg1 protein, or an active fragment or homolog thereof, prior to or at the time of antibiotic administration in order to provide the intestinal system with protection against infectious and/or inflammatory diarrhea. For example, Reg1 protein can be administered intravenously, intramuscularly, orally or by enema.

One embodiment provides a method to treat infectious and/or inflammatory diarrhea comprising administering to a subject in need thereof an effective amount of at least one Reg1 protein, or an active homolog, variant or fragment thereof.

Another embodiment provides a prophylactic treatment of infectious and/or inflammatory diarrhea comprising administering to a subject an effective amount of at least one Reg1 protein, or an active homolog, variant or fragment thereof.

The present invention provides for the treatment of infectious and/or inflammatory diarrhea by inducing the expression of Reg1 or by administering Reg1 protein, or an active homolog or fragment thereof, or an isolated nucleic acid comprising a sequence encoding a Reg1 protein or an active fragment or homolog thereof.

The present application is the first to describe a role of Reg1 in defense against infectious and/or inflammatory diarrhea, including, but not limited to, C. difficile. The availability of reagents to detect the expression of Reg1, and of purified Reg1, makes application of the invention feasible and immediate.

Various aspects and embodiments of the invention are described in further detail below.

### BRIEF DESCRIPTION OF THE DRAWINGS

FIG. 1. Wild type mouse pretreated with antibiotics and then infected with C. difficile. Note the intact epithelium with modest submucosal inflammation.

FIG. 2. Reg1 −/− mouse pretreated with antibiotics and then infected with C. difficile. Note the extensive intestinal epithelial disruption and inflammation.

FIG. 3. Cecal pathology score for Reg1 −/− (Reg1 KO) and wild type (WT) mice pretreated with antibiotics and then infected with C. difficile. Note the significantly higher pathologic score for the Reg1 −/− mice.

FIG. 4. Survival curve for Reg1 −/− and wild type mice

FIG. 5. Comparison between the susceptibility of REG1 −/−, WT, and heterozygote (+/−) mice to infection with E. histolytica. There was a significant difference in susceptibility to E. histolytica infection in REG1 −/− and wild type mice (*p=0.02), and between REG1 −/+ and wild type mice (**p=0.04), suggesting that having two copies of the REG1 gene is important for resistance to E. histolytica infection.

FIG. 6. SDS-PAGE and Coomassie Blue stained purified REG1A-GST and REG1B-GST.

FIG. 7. SDS-PAGE and Coomassie Blue-stained proteins in samples taken during Reg1 protein purification. Lanes: 1 Molecular weight standards (kDaltons), 2 cell lysates, 3 initial inclusion body (IB) pellet, 4 IB after first wash, 5 IB after second wash, 6 solubilized IB, 7 purified Reg1A(arrow), 8 purified Reg1B.

FIG. 8. Immunoblot of (A) Reg1A and (B) Reg1B purified by described Method. Left lanes contain molecular weight standards. Other lanes contain 75, 50, 25 and 1 ng Reg1 protein. Blots were developed with rat anti-Reg1A-specific monoclonal antibody (R&D Systems, 1:5000 dilution) or goat anti-Reg1B-specific polyclonal antibody (R&D Systems, 1:100 dilution) followed by HRP-conjugated anti-rat or HRP-conjugated anti-goat antibody.

FIG. 9. ELISA assay standard curves for Reg1B and Reg1A.

FIG. 10. Immunoblot of Reg1A in human fecal samples. Lane 1 displays molecular weight standards (kDaltons). Lane 2 is 0.5 micrograms recombinant human Reg1A (upper arrow). Lanes 3-7 are human fecal samples processed as in Methods (lower arrow indicates cleaved Reg1A).

FIG. 11. Comparison by immunohistochemistry of REG1 expression in a patient with intestinal inflammation due to C. difficile vs a patient with normal histology. A. REG1A staining in a colonic biopsy sample with C. difficile colitis and intestinal inflammation. B. REG1A staining in a colonic biopsy sample with normal histology (C. difficile status unknown)

FIG. 12A-E. REG1 immunohistochemical staining (IHC) of human colonic biopsy samples during acute E. histolytica diarrhea (day 1) and convalescence (day 60). A) REG1A staining of an ulcer region during acute E. histolytica diarrhea. B) REG1A during convalescence. C) REG1B staining of an ulcer region during acute E. histolytica diarrhea. D) REG1B during convalescence. E. Reg1B ELISA results of the same human patient and healthy donor fecal samples as in FIG. 10, Lanes 3-7.

FIG. 13. Results of ELISA assays for C. difficile antigen, human lactoferrin, and human Reg1B in patient fecal samples. Samples were from patients both with and without C. difficile-associated disease.

FIG. 14. Detection of Reg1A by immunoblot and Reg1B, C. difficile antigen, and human lactoferrin by ELISA in human fecal samples. The two right-most samples contain data from FIGS. 10 and 12.

### DETAILED DESCRIPTION OF THE INVENTION

Described herein is a method for extraction, detection and quantification of Reg1 proteins in feces, such as by immunoassay, for use in diagnosing inflammatory diarrhea, including, but not limited to diarrhea caused by Clostridium difficile (C. diff). Patients are further monitored using Reg1 protein detection/quantification to evaluate the effectiveness of medical therapy and predict relapse. In one embodiment, the detection and/or quantification is carried out by immunoassay.

### Definitions

As used herein, the terms below are defined by the following meanings:

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Plurality” means at least two.

A “subject” is a vertebrate, including a mammal, such as a human. Mammals include, but are not limited to, humans, farm animals, sport animals and pets.

The term “biological sample,” as used herein, refers to samples obtained from a subject, including, but not limited to, skin, hair, tissue, blood, plasma, serum, cells, sweat, saliva, feces, tissue and/or urine.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

As used herein, the term “biologically active fragments” or “bioactive fragment” of the polypeptides encompasses natural or synthetic portions of the full length protein that are capable of specific binding to their natural ligand or of performing the function of the protein. For example, a “functional” or “active” biological molecule is a biological molecule in a form in which it exhibits a property by which it is characterized. A functional enzyme, for example, is one which exhibits the characteristic catalytic activity by which the enzyme is characterized.

A “fragment” or “segment” is a portion of an amino acid sequence, comprising at least one amino acid, or a portion of a nucleic acid sequence comprising at least one nucleotide. The terms “fragment” and “segment” are used interchangeably herein. As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length. As used herein, the term “fragment” as applied to a nucleic acid, may ordinarily be at least about 20 nucleotides in length, typically, at least about 50 nucleotides, more typically, from about 50 to about 100 nucleotides, at least about 100 to about 200 nucleotides, at least about 200 nucleotides to about 300 nucleotides, at least about 300 to about 350, at least about 350 nucleotides to about 500 nucleotides, at least about 500 to about 600, at least about 600 nucleotides to about 620 nucleotides, at least about 620 to about 650, and or the nucleic acid fragment will be greater than about 650 nucleotides in length.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands. “Binding partner,” as used herein, refers to a molecule capable of binding to another molecule.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “isolated” refers to a compound, including antibodies, nucleic acids or proteins/.peptides, or cell that has been separated from at least one component which naturally accompanies it.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “substantially pure” describes a compound, e.g., a protein or polypeptide, cell or nucleic acid that has been separated from components which naturally accompany it. Typically, a compound is substantially pure when at least 10%, including at least 20%, at least 50%, at least 60%, at least 75%, at least 90%, at least 95%, at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “compound,” as used herein, refers to any type of substance or agent that is can be considered a drug, or a candidate for use as a drug, as well as combinations and mixtures of the above.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. The term “regulate” refers to either stimulating or inhibiting a function or activity of interest.

An “effective amount” generally means an amount which provides the desired local or systemic effect, such as enhanced performance. For example, an effective dose is an amount sufficient to affect a beneficial or desired clinical result. The dose could be administered in one or more administrations and can include any preselected amount.

The precise determination of what would be considered an effective dose may be based on factors individual to each subject, including size, age, injury or disease being treated and amount of time since the injury occurred or the disease began. One skilled in the art, particularly a physician, would be able to determine what would constitute an effective dose.

“Co-administer” can include simultaneous and/or sequential administration of two or more agents.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As use herein, the terms “administration of” and or “administering” a compound should be understood to mean providing a compound of the invention or a prodrug of a compound of the invention to a subject in need of treatment.

As used herein, “treat,” “treating” or “treatment” includes treating, ameliorating, or inhibiting an injury or disease related condition or a symptom of an injury or disease related condition. In one embodiment the disease, injury or disease related condition or a symptom of an injury or disease related condition is prevented; while another embodiment provides prophylactic treatment of the injury or disease related condition or a symptom of an injury or disease related condition. The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

As used herein, “health care provider” includes either an individual or an institution that provides preventive, curative, promotional or rehabilitative health care services to a subject, such as a patient.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, “alleviating a disease or disorder symptom,” means reducing the severity of the symptom or the frequency with which such a symptom is experienced by a patient, or both. A “therapeutic” treatment is a treatment administered to a subject who exhibits signs of pathology for the purpose of diminishing or eliminating those signs.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

“Pharmaceutically acceptable” means physiologically tolerable, for either human or veterinary application.

As used herein, “pharmaceutical compositions” include formulations for human and veterinary use.

The term “standard,” as used herein, refers to something used for comparison. For example, it can be a known standard agent or compound which is administered and used for comparing results when administering a test compound or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker. Standard can also refer to a healthy individual.

As used herein, an “instructional material” includes a publication, a recording, a diagram, or any other medium of expression which can be used to communicate the usefulness of the peptide or antibody of the invention in the kit for diagnosing or effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The terms “comprises”, “comprising”, and the like can have the meaning ascribed to them in U.S. Patent Law and can mean “includes”, “including” and the like. As used herein, “including” or “includes” or the like means including, without limitation.

### Diarrhea

Inflammatory diarrhea occurs when there is damage to the intestinal mucosal lining or brush border, which leads to a passive loss of protein-rich fluids, and a decreased ability to absorb these lost fluids. Features of all three of the other types of diarrhea can be found in this type of diarrhea. It can be caused by bacterial infections, viral infections, parasitic infections, or autoimmune problems such as inflammatory bowel diseases. It can also be caused by tuberculosis, colon cancer, and enteritis.

Inflammatory diarrheas include those caused by enteric pathogens including, but not limited to, Campylobacter jejuni, Salmonella species, Shigella species, Escherichia coli (including enterohemorrhagic, enterotoxigenic, enteroaggregative E. coli), Entamoeba histolytica, Clostridium difficile, Cryptosporidium and those that have no clearly defined infectious agent such as, Crohn's Disease (CD) and ulcerative colitis (UC).

Clostridium difficile antibiotic-associated colitis is an increasing problem in health-care associated diarrhea, made more so recently by the emergence of a quinolone-resistant hyper-virulent strain. The infection is potentially fatal, difficult to treat, and prone to relapse.

Infectious diarrhea or contagious diarrhea may be defined as diarrhea caused by an infection of the digestive system by a bacterium, virus, or parasite that result in frequent bowel motions producing liquid feces. Viral diarrheas include, but are not limited to, those caused by Norovirus, Rotavirus, Adenovirus, or Astrovirus. Bacterial diarrheas, including diarrheas caused by their toxins, include, but are not limited to, diarrhea caused by Campylobacter jejuni, Salmonella, Shigella, Vibrio cholerae/Cholera, Vibrio parahaemolyticus, Escherichia coli (including enterohemorrhagic, enterotoxigenic, enteroaggregative E. coli), Clostridium difficile, staphylococcal toxin and Bacillus cereus.

### Regenerating Protein 1 (REG)

Amounts of Reg protein 1 have been shown to be stimulated during the regeneration of pancreatic islets. Since then, many Reg-related proteins have been identified in humans and other animals. In human, several REG family genes, including REG1 alpha, REG1 beta, REG-related sequence (RS) and HIP/PAP, have been isolated. These Reg-related proteins are classified into subfamilies according to their amino-acid sequences, but they share a similar structure. Regenerating proteins 1A and B are sometimes referred to by different names, including lithostathine-1-alpha and lithostathine-1-beta or pancreatic stone protein. These are proteins that in humans are encoded by the REG1A gene or REG1B gene [46, 47, 48, and 49].

The mRNA and protein sequence of Reg1A (lithostathine-1-alpha) is provided, for example, at Genbank accession no. NM_{—}002909.4 and NP_{—}002900.2, respectively:

| (SEQ ID NO: 1) | |
|---|---|
| gatataaagc tcctacagct acctggcctg agaagccaac tcagactcag ccaacagaga | 61 |
| ttgttgattt gcctcttaag caagagattc attgcagctc agcatggctc agaccagctc | 121 |
| atacttcatg ctgatctcct gcctgatgtt tctgtctcag agccaaggcc aagaggccca | 181 |
| gacagagttg ccccaggccc ggatcagctg cccagaaggc accaatgcct atcgctccta | 241 |
| ctgctactac tttaatgaag accgtgagac ctgggttgat gcagatctct attgccagaa | 301 |
| catgaattcg ggcaacctgg tgtctgtgct cacccaggcc gagggtgcct ttgtggcctc | 361 |
| actgattaag gagagtggca ctgatgactt caatgtctgg attggcctcc atgaccccaa | 421 |
| aaagaaccgc cgctggcact ggagcagtgg gtccctggtc tcctacaagt cctggggcat | 481 |
| tggagcccca agcagtgtta atcctggcta ctgtgtgagc ctgacctcaa gcacaggatt | 541 |
| ccagaaatgg aaggatgtgc cttgtgaaga caagttctcc tttgtctgca agttcaaaaa | 601 |
| ctagaggcaa ctggaaaata catgtctaga actgatccag caattacaac ggagtcaaaa | 661 |
| attaaaccgg accatctctc caactcaact caacctggac actctcttct ctgctgagtt | 721 |
| tgccttgtta atcttcaata gttttaccta ccccagtctt tggaacccta aataataaaa | 781 |
| ataaacatgt ttccactatt gtgctgtcaa aaaaaaaaaa a | |

```
(SEQ ID NO: 2)
    1 maqtssyfml isclmflsqs qgqeaqtelp qariscpegt nayrsycyyf nedretwvda
  61 dlycqnmnsg nlvsvltqae gafvaslike sgtddfnvwi glhdpkknrr whwssgslvs
121 ykswgigaps svnpgycvsl tsstgfqkwk dvpcedkfsf vckfkn.
```

The mRNA and protein sequence of Reg1B (lithostathine-1-beta) is provided, for example, at Genbank accession no. NM_{—}006507.3 and NP_{—}006498, respectively:

| (SEQ ID NO: 3) | |
|---|---|
| gatataaagc tcctactctg tctgacctga caagccacct caagtggaca aggcacttac | 61 |
| caacagagat tgctgatttg ctccttaagc aagagattca ctgccgctaa gcatggctca | 121 |
| gaccaactcg ttcttcatgc tgatctcctc cctgatgttc ctgtctctga gccaaggcca | 181 |
| ggagtcccag acagagctgc ctaatccccg aatcagctgc ccagaaggca ccaatgccta | 241 |
| tcgctcctac tgctactact ttaatgaaga ccctgagacc tgggttgatg cagatctcta | 301 |
| ttgccagaac atgaattcag gcaacctggt gtctgtgctc acccaggcgg agggtgcctt | 361 |
| cgtggcctca ctgattaagg agagtagcac tgatgacagc aatgtctgga ttggcctcca | 421 |
| tgacccaaaa aagaaccgcc gctggcactg gagtagtggg tccctggtct cctacaagtc | 481 |
| ctgggacact ggatccccga gcagtgctaa tgctggctac tgtgcaagcc tgacttcatg | 541 |
| ctcaggattc aagaaatgga aggatgaatc ttgtgagaag aagttctcct ttgtttgcaa | 601 |
| gttcaaaaac tagaggaagc tgaaaaatgg atgtctagaa ctggtcctgc aattactatg | 661 |
| aagtcaaaaa ttaaactaga ctatgtctcc aactcagttc agaccatctc ctccctaatg | 721 |
| agtttgcatc gctgatcttc agtaccttca cctgtctcag tctctagagc cctgaaaaat | 781 |
| aaaaacaaac ttatttttat ccagtgaaaa aa | |

```
(SEQ ID NO: 4)
    1 maqtnsffml isslmflsls qgqesqtelp npriscpegt nayrsycyyf nedpetwvda
  61 dlycqnmnsg nlvsvltqae gafvaslike sstddsnvwi glhdpkknrr whwssgslvs
121 ykswdtgsps sanagycasl tscsgfkkwk descekkfsf vckfkn.
```

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul [50; 1990]), modified as in Karlin and Altschul [51; 1993]. This algorithm is incorporated into the NBLAST and XBLAST programs of Altschul, et al. [52], and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. [53]. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, a “substantially homologous amino acid sequences” or “substantially identical amino acid sequences” includes those amino acid sequences which have at least about 92%, or at least about 95% homology or identity, including at least about 96% homology or identity, including at least about 97% homology or identity, including at least about 98% homology or identity, and at least about 99% or more homology or identity to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

“Substantially homologous nucleic acid sequence” or “substantially identical nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In one embodiment, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 92%, 95%, 99% or more. Substantial identity of nucleic acid sequences can be determined by comparing the sequence identity of two sequences, for example by physical/chemical methods (i.e., hybridization) or by sequence alignment via computer algorithm.

Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package [54, 55, 52, and 53]. The default settings provided with these programs are suitable for determining substantial similarity of nucleic acid sequences for purposes of the present invention.

Reg1A or B protein can be obtained by recombinant and/or purification methods available to the art worker or can be purchased commercially. Biologically active variants, mutations, fragments or substantially homologous or identical amino acid or nucleic acid sequences, or combinations thereof, of Reg1A or B are also within the scope of the invention.

### Detection

In one embodiment, the detection and/or quantification of Reg1A or B is carried out by an immunoassay. An immunoassay is a biochemical test that measures the presence or concentration of a substance in solutions that frequently contain a complex mixture of substances. Analytes in biological samples, such as feces, serum or urine are frequently assayed using immunoassay methods. Such assays are based on the unique ability of an antibody to bind with high specificity to one or a very limited group of molecules. A molecule that binds to an antibody is called an antigen. Immunoassays can be carried out for either member of an antigen/antibody pair. In either case the specificity of the assay depends on the degree to which the analytical reagent is able to bind to its specific binding partner to the exclusion of all other substances that might be present in the sample to be analyzed. In addition to the need for specificity, a binding partner must be selected that has a sufficiently high affinity for the analyte to permit an accurate measurement. The affinity requirements depend on the particular assay format that is used.

In addition to binding specificity, the other feature of all immunoassays is a means to produce a measurable signal in response to a specific binding. Most immunoassays depend on the use of an analytical reagent that is associated with a detectable label. A large variety of labels have been demonstrated including radioactive elements used in radioimmunoassays; enzymes; fluorescent, phosphorescent, and chemiluminescent dyes; latex and magnetic particles; dye crystallites, gold, silver, and selenium colloidal particles; metal chelates; coenzymes; electroactive groups; oligonucleotides, stable radicals, and others. Such labels serve for detection and quantitation of binding events either after separating free and bound labeled reagents or by designing the system in such a way that a binding event effects a change in the signal produced by the label.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

Immunoassays include, but are not limited to, Enzyme-linked immunosorbent assay (ELISA), lateral flow test, latex agglutination, other forms of immunochromatography, western blot, Luminex, QuikChek, and/or magnetic immunoassay.

Techniques useful for practicing the invention are described herein (see Figures) or are known in the art. Other useful ELISA assays and reagents for detecting Reg1A and Reg1B include: Reg1B assay kit (catalog # E94674Hu-ELISA Kit for Human Regenerating Islet Derived Protein 1 Beta (Reg1b); Uscn Life Science Inc. Wuhan); recombinant human Reg1B (catalog #11638-H08H, Sino Biological, Inc. or catalog #H00005968-P01, Novus Biologicals); recombinant Reg1A (catalog #11234-H08H, Sino Biological, Inc.); Reg1A antibody for ELISA (catalog #11234-RP01; Sino Biological, Inc.).

Also included are methods to detect Reg1 mRNA in, for example, intestinal biopsies or feces. These detection methods include, but are not limited to, Northern blot analysis, nuclease protection assays (NPA), in situ hybridization, and/or reverse transcription-polymerase chain reaction (RT-PCR). Based on the known sequences of Reg1 mRNA, including Reg1A and Reg1B, one of skill in the art can easily make use of available mRNA detection techniques to detect Reg1 mRNA in a sample.

### Computer/Processor

The detection and/or diagnosis method can employ the use of a processor/computer system. For example, a general purpose computer system comprising a processor coupled to program memory storing computer program code to implement the method, to working memory, and to interfaces such as a conventional computer screen, keyboard, mouse, and printer, as well as other interfaces, such as a network interface, and software interfaces including a database interface find use one embodiment described herein.

The computer system accepts user input from a data input device, such as a keyboard, input data file, or network interface, or another system, such at the system interpreting, for example, the ELISA data, and provides an output to an output device such as a printer, display, network interface, or data storage device. Input device, for example a network interface, receives an input comprising detection of the proteins described herein and/or quantification of those proteins. The output device provides an output such as a display, including one or more numbers and/or a graph depicting the detection and/or quantification of the proteins.

Computer system is coupled to a data store which stores data generated by the methods described herein. This data is stored for each measurement and/or each subject; optionally a plurality of sets of each of these data types is stored corresponding to each subject. One or more computers/processors may be used, for example, as a separate machine, for example, coupled to computer system over a network, or may comprise a separate or integrated program running on computer system. Whichever method is employed these systems receive sequence data and provide data regarding detection/diagnosis in return.

### Antibodies/Detection Agents

Antibodies to detect Reg1A or B proteins can be produced by methods available to an art worker or purchased commercially.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin subunit molecules. The antibodies in the present invention may exist in a variety of forms including, for example, polyclonal antibodies, monoclonal antibodies, Fv, Fab and F(ab)2, as well as single chain antibodies and humanized antibodies.

As used herein, the term “secondary antibody” refers to an antibody that binds to the constant region of another antibody (the primary antibody).

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

### Treatment/Prevention of Disease/Disorder

The present application is the first to describe a role of Reg1 in defense against infectious and/or inflammatory diarrhea, including, but not limited to, C. difficile. The availability of reagents to detect Reg1 protein or mRNA and of method s to produce and administer therapeutic amounts of Reg1 protein makes application of the invention feasible and immediate.

A Reg1 protein or a variant, homolog, or active fragment or nucleic acid encoding Reg1 protein or a variant, homolog, or active fragment thereof, and optionally at least one additional antimicrobial and/or antidiarrheal agent, can be formulated as a pharmaceutical composition and administered to a mammalian host, such as a human patient in a variety of forms adapted to the chosen route of administration, i.e., topical, oral, buccal, intravenous, intramuscular, intra arterial, intramedullary, intrathecal, intraventricular, parenteral (subcutaneous, intraperitoneal, intramuscular, intrasternal injection, and kidney dialytic infusion techniques), transdermal, subcutaneous, intraperitoneal, intranasal, enteral, topical, sublingual, vaginal, ophthalmic, pulmonary, or rectal means (such as by enema or suppository). As Reg1 proteins are made by cells lining the intestinal tract and are secreted into the lumen of the intestines, Reg1 protein administered orally or rectally for defense against or treatment of diarrhea places this protein within its natural setting.

An anti-diarrhoeal drug (or anti-diarrheal drug) is any medication which provides symptomatic relief for diarrhea. These include electrolyte solutions, bulking agents (e.g., methylcellulose, guar gum or plant fibre (bran, sterculia, isabgol etc.), absorbents (to absorb toxic substances that cause infective diarrhea, for example, methylcellulose is an abosorbent), anti-inflammatory agents such as bismuth subsalicylate, or opiods (e.g., morphine or codeine, loperamide).

Antimicrobials are substances that kill or inhibit the growth of microorganisms such as bacteria, fungi, virus or protozoans. Antimicrobial drugs either kill microbes (microbiocidal) or prevent the growth of microbes (microbiostatic).

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices. For example the, the active compound can be formulated so as to release only in the intestine and/or the colon.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary with the route of administration, the nature of the condition being treated and the age and condition of the patient and will be ultimately at the discretion of the attendant physician or clinician.

### EXAMPLES

The following examples are provided in order to demonstrate and further illustrate certain embodiments and aspects of the present invention and are not to be construed as limiting the scope thereof.

### Example 1

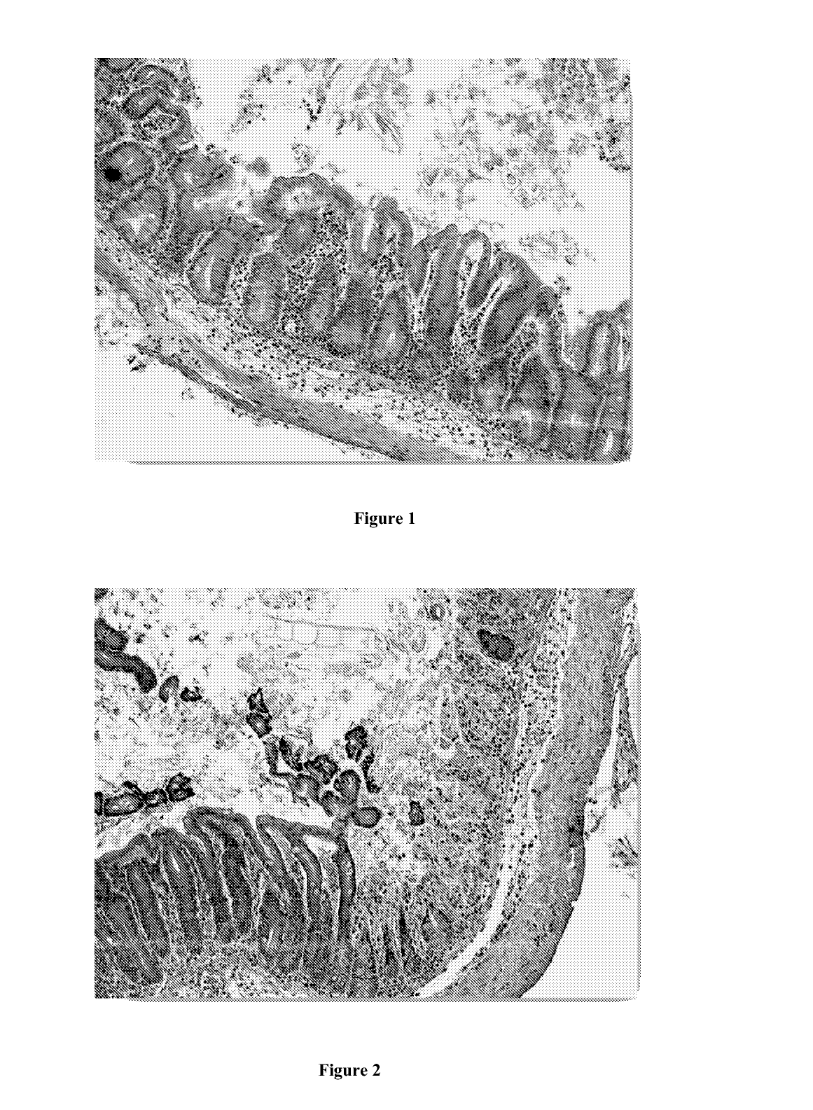

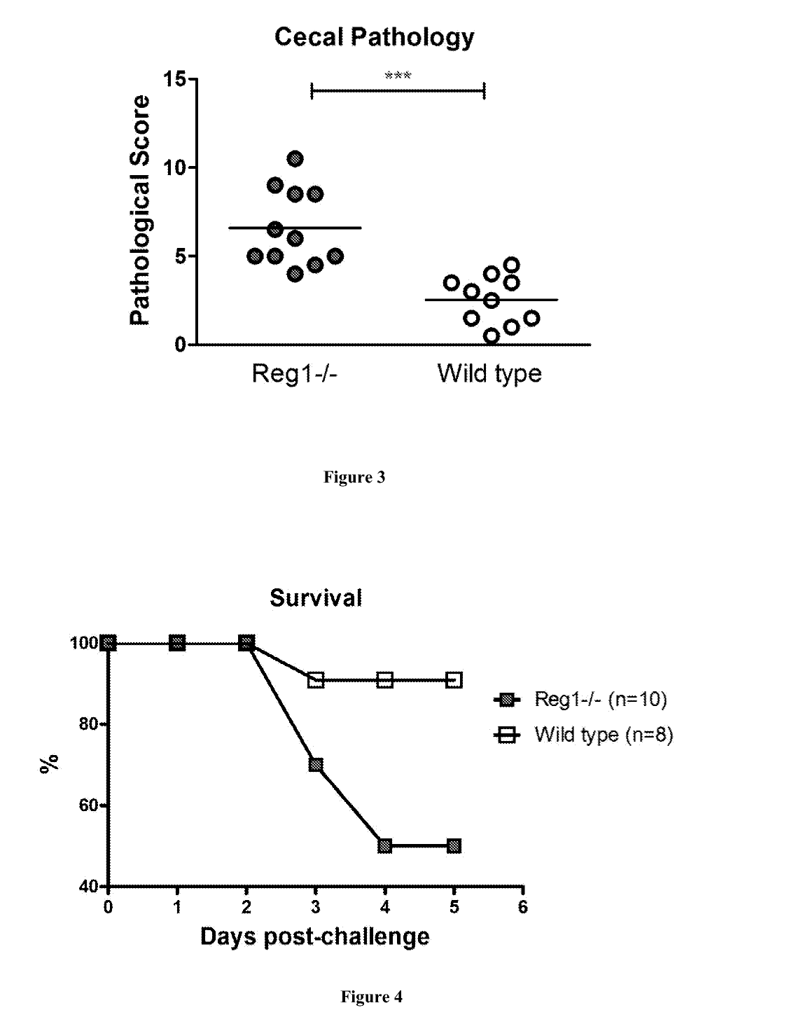

The present invention is based on the discovery described herein that mice genetically deficient in Reg1 have increased susceptibility and severity of Clostridium difficile infection. Wild type mice were pretreated with antibiotics and then infected with C. difficile. Note in the micrograph of FIG. 1 the intact epithelium with modest submucosal inlammation in the wild type mice. Reg1 −/− mice were pretreated with antibiotics and then infected with C. difficile. Note in the micrograph of FIG. 2 the extensive intestinal epithelial disruption and inflammation in the Reg1 −/− mice. FIG. 3 graphically illustrates cecal pathology scores for Reg1 −/− (Reg1 KO) and wild type (WT) mice pretreated with antibiotics and then infected with C. difficile. Note the significantly higher pathologic score for the Reg1 −/− mice. It can be seen in FIG. 4 that survival was greatest in wild type mice.

### Example 2

### Colonic Biopsy Samples

Colon biopsy samples for microarray, RT-qPCR, and immunohistochemistry (IHC) were obtained from eight patients, ranging in age from 17 to 40, who came to the

International Centre for Diarrhoeal Diseases, Bangladesh, with acute E. histolytica diarrhea. Patients with acute disease were stool antigen positive for E. histolytica, had blood and mucous in their stools, and were culture negative for other common diarrheal agents. Colonic biopsies were obtained from an ulcerative region of the sigmoid colon in subjects with an acute episode of E. histolytica diarrhea, and the normal colon 60 days later during convalescence. Biopsy specimens were placed in RNAlater (Qiagen, CA) and stored at −80° C. until isolation of RNA for microarray and RT-qPCR, or collected in Histocon (Histolab, Gutenberg, Sweden) and snap frozen in liquid nitrogen for IHC.

### Microarray

Samples were prepared according to Affymetrix protocols (Affymetrix, Inc., Santa Clara, Calif.). RNA quality and quantity was ensured using the Bioanalyzer (Agilent, Inc) and NanoDrop (Thermo Scientific, Inc) respectively. Per RNA labeling, 300 nanograms of total RNA was used in conjunction with the Affymetrix recommended protocol for the GeneChip 1.0 ST chips. The hybridization cocktail containing the fragmented and labeled cDNAs were hybridized to The Affymetrix Human GeneChip® 1.0 ST chips. The chips were washed and stained by the Affymetrix Fluidics Station using the standard format and protocols as described by Affymetrix. The probe arrays were stained with streptavidin phycoerythrin solution (Molecular Probes, Carlsbad, Calif.) and enhanced by using an antibody solution containing 0.5 mg/mL of biotinylated anti-streptavidin (Vector Laboratories, Burlingame, Calif.). An Affymetrix Gene Chip Scanner 3000 was used to scan the probe arrays. Gene expression intensities were calculated using GeneChip® Command Console® Software (AGCC)<http://www.affymetrix.com/products_services/software/specific/command_console_software.affx> and Expression Console™ Software <http://www.affymetrix.com/products_services/software/specific/expression_console_s oftware.affx>. Cel files generated by the Affymetrix AGCC program were imported in the Affymetrix Expression Consol software and RMA (Robust Multichip Analysis) normalization was performed to generate the .Chp files. The .Chp files are normalized, log2 transformed and summarized. Pairwise comparisons within GeneSifter software (VizX Labs, Seattle, Wash.) were performed on the .Chp files and t-test and Benjamin and Hochberg correction were applied.

### Real-Time Polymerase Chain Reaction.

Total RNA from colonic tissue was isolated using TRIzol reagent (Invitrogen, Carlsabad, Calif.) and reverse transcribed into cDNAs using Superscript II reverse transcriptase. Real-time PCR was performed in duplicate using a standard Quantifast SYBR Green protocol per manufacturer's instructions, Quantifast PCR master mix, and Quantitect PCR primers for REG1A and REG1B and the housekeeping genes Peptidylprolyl isomerase A (PPIA) and hypoxanthine phosphoribosyltransferase (HPRT1) (Qiagen, Valencia, Calif.).

A mixed model repeated measure (MMRM) with a compound symmetry covariance structure was performed using all available C(t) values. The model includes a variable of gene (4 levels for the 4 genes), a variable of day (2 levels for day 1 and day 60) and the interaction between the two variables. This regression model accounts for the correlation between C(t) measures from the same biopsy samples for the 4 genes and between the duplicates. Contrasts were constructed to estimate the means and standard errors of the means for the normalized C(t) for the two study genes, REG1A and REG1B, at day 1 and day 60 and to examine the expression difference between the two study genes.

### Immunohistochemical Staining

Immunohistochemistry was performed for the detection of REG1A and REG1B in colonic biopsy samples. Tissue sections were deparaffined and rehydrated prior to antigen retrieval using the Pascal Pressure Chamber with TRS buffer for 30 seconds at 125° C. and 22 psi. Slides were stained using the Dako Autostainer Universal Staining system (DAKO, Denmark). Sections were incubated for 10 minutes with DAKO Dual Endogenous Enzyme Block prior to a 30-minute incubation with the primary antibody, either Rat anti-human REG 1A monoclonal antibody at a 1:150 dilution, or goat anti-human REG1B polyclonal antibody at a 1:200 dilution (R&D systems, Minneapolis, Minn.). Sections were subsequently incubated for 30 minutes with rabbit anti-rat biotinylated antibody at 1:200 dilution (DakoCytomation) or anti-goat biotinylated antibody (Vector laboratories, Burlingame, Calif.) respectively. The sections were then incubated with a 1:200 dilution of horseradish peroxidase streptavidin (Vector laboratories) prior to incubation with chromagen substrate diaminobenzidine tetrahydrochloride (DAB) for 10 minutes. The slides were counterstained with hematoxylin and mounted. Negative controls were performed by omitting the primary antibody. Colon tissue from subjects with ulcerative colitis was used as a positive control. IgG isotype controls were additionally performed.

### Animals

Regenerating gene 1 (REG1) knock-out mice in the ICR (CD1) background were previously generated by Unno et al [27] by disruption of the exon 2 of REG1 gene with a targeting vector and subsequent germ-line transmission in chimeric mice. Brother-sister mating of REG1 +/− mice was carried out to generate REG1 −/− and wild type mice.

### Animal Model for Intestinal Amebic Infection

To infect each mouse, 2×10⁶ E. histolytica trophozoites were injected intracecally after laparotomy. Mice were then sacrificed at 4 days post-challenge. The cecum was harvested and rinsed with 500 μl PBS, and cecal rinse was cultured in complete TYI-S-33 medium with supplemental antibiotics for 5 days at 37° C. Infection status was determined by amebic culture positivity.

### Microarray

Colon biopsies from patients with acute E. histolytica colitis were obtained from the sigmoid colon in an area of ulceration. Convalescent biopsies for comparison were obtained from the same patient 60 days later. Gene transcripts were analyzed using the Affymetrix Human GeneChip® 1.0 ST chips. REG1A and REG1B were the most upregulated genes in the human intestine during acute E. histolytica disease compared to convalescence (p=<0.003 and p=0.006 respectively) (Table 1).

TABLE 1
RNA from colonic biopsy samples was isolated simultaneously from the same sample
for microarray and RT-qPCR. (A) Comparison of microarray and (B) RT-qPCR REG 1A
and REG 1B mRNA expression between colon biopsy samples from individuals with acute
E. histolytica diarrhea (day 1) versus convalescence (day 60).
(A)

| | Day 1 mean | Day 60 mean | | |
|---|---|---|---|---|
| | expression | expression | Fold | |
| | (Log2) (±SEM) | (Log2) (±SEM) | change | p-value |
| REG 1A | 10.69 (0.76) | 7.8 (0.22) | 7.4 | 0.003 |
| REG 1B | 9 (1.02) | 5.59 (0.23) | 10.65 | 0.006 |

(B)

| | Day 1 | Day 60 | | | |
|---|---|---|---|---|---|
| | normalized | normalized | CT change | Expression | |
| | CT mean | CT mean | mean | Ratio | |
| | (±SEM) | (±SEM) | (SEM) | (Day 1/Day 60) | p-value |
| REG 1A | 3.93 (0.86) | 9.98 (0.96) | −6.05 (1.29) | 66.3 | <0.001 |
| REG 1B | 4.07 (0.86) | 9.04 (0.96) | −4.97 (1.29) | 31.3 | 0.001 |

### PCR

RNA from colonic biopsy samples was isolated simultaneously from the same sample for microarray and PCR. Quantitative PCR confirmed the microarray results that REG1A and REG1B expression are significantly increased in colonic biopsy samples during acute E. histolytica disease compared to convalescence (p=<0.001 and p=0.001 respectively) (Table 1). The expression ratio for day 1 over day 60 samples was 66.3 for REG1A and 31.3 for REG1B.

### Immunohistochemistry

REG1A and REG1B staining was observed in the epithelial cells of the intestinal crypts during acute E. histolytica disease. The staining was nearly absent during convalescence (FIG. 12). The staining was most intense in the crypts with a gradient decreasing as the cells migrated to the apical surface. Predominant staining of the intestinal crypt epithelial cells during inflammatory colitis has also been noted by others. REG1A and REG1B staining were not detected in the negative controls when primary antibody was excluded, or with the IgG isotype controls (data not shown).

REG1 was associated with protection from the development of E. histolytica infection in vivo

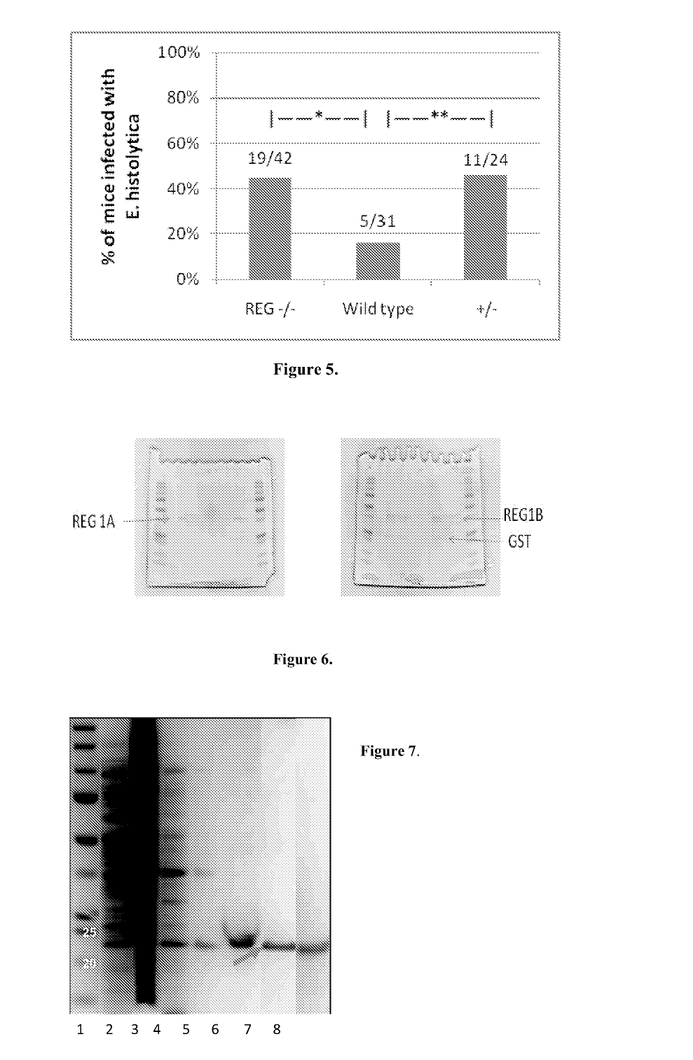

To determine if REG1 had a role in protection from E. histolytica infection, REG1 −/−, REG1 −/+, and wild type mice were challenged by intracecal injection of E. histolytica. Four days post-challenge the mice were sacrificed and cecal content culture was performed. There was a significant difference in susceptibility to E. histolytica infection between REG1 −/− and wild type mice (p=0.02), and between REG1 −/+ and wild type mice (p=0.04) (FIG. 5), suggesting that having two copies of the REG1 gene was important for resistance to E. histolytica infection.

In the current study, REG1A and REG1B staining was intense during the acute stage of E. histolytica colitis and present throughout the intestinal cells of the crypt epithelium. In contrast, both REG1A and REG1B staining were limited to the basal portion of the colonic crypts after recovery. These findings correspond with the microarray and PCR data showing markedly increased REG1A and REG1B expression during acute E. histolytica colitis. REG1A, but not REG1B, staining was also detectable in the lamina propria of both acute and convalescent samples. REG1A and REG1B staining were not detected in the negative controls when primary antibody was excluded, or with the IgG isotype controls.

In summary, REG1A and REG1B mRNA and protein expression were significantly increased in the human intestine during acute E. histolytica colitis. Increased REG1 expression may be beneficial during E. histolytica colitis through a protective role.

### Example 3

Colonic Biopsy Samples for Evaluation of Clostridium difficile

Colonic biopsy samples were obtained from 5 patients with a positive C. difficile toxin assay and 2 patients with an unknown, but presumed negative, C. difficile status. The biopsies were evaluated by immunohistochemistry for REG1A and REG1B using the methods as described in the previous section.

Animal Model for Intestinal Clostridium difficile Infection

Mice were challenged with an oral inoculum of the VPI 10463 strain of C. difficile at two different doses, either 10⁵ or10⁶ cfu. They were monitored for change in body weight from day 0 to day 6, and were sacrificed at six days post-challenge. Tissue was prepared for histology, and infection detected by cecal antigen and cecal content culture. The histological severity of enteritis was graded taking into account the following features: epithelial cell destruction, mononuclear cell infiltration, and edema of the submucosa.

Expression and Purification of REG1A and REG1B Proteins with a GST tag

Clones with full length REG1A and REG1B genes were obtained from Invitrogen, and primers were designed for REG1A and REG1B to produce blunt-end PCR products using these clones. TOPO cloning was performed with the PCR products and an entry vector (pENTR™/SD/D-TOPO®, Invitrogen), and this vector was transformed into competent E. coli. Plasmid DNA was isolated from colonies and sequenced for confirmation prior to combining with a destination vector (Gateway® pDEST™ 15, Invitrogen) which provided a GST tag. The recombination product was then transformed into competent DH5a cells. Expression clones were selected and sequenced to confirm the REG1A and REG1B genes were cloned in frame. Purified plasma DNA of the expression clone was transformed into BL21-AI™ One Shot® cells (Invitrogen) prior to induction of REG1A and REG1B protein expression. Since the overexpressed proteins contained a GST tag, Glutathione sepharose beads (Amersham Pharmaceuticals) were used to purify the proteins. SDS-polyacrylamide gel electrophoresis (SDS-PAGE) of the purified proteins stained with Coomassie Blue showed proteins of the correct size (42 kDalton). The only other visible contaminant protein was consistent with GST (26 kDalton) (FIG. 6).

Expression and Purification of Reg1A and Reg1B Proteins with a His₆ Tag in E. coli

Bacterial protein expression vectors (pReceiver-B01) expressing amino acids 22-166 of human Reg1A or Reg1B in which the mammalian signal sequence was removed and replaced with a leader encoding an amino-terminal hexa-histidine tag were purchased from GeneCopoeia, Inc., Rockville, Md. 20850,USA. Vector DNA was introduced into BL21 Star (DE3) pLysS bacterial cells (Invitrogen); colonies were selected with antibiotic and further grown in liquid growth cultures. Protein production was induced by treatment with 0.4 mM IPTG (isopropyl β-D-1-thiogalactopyranoside) for 3-4 hours.

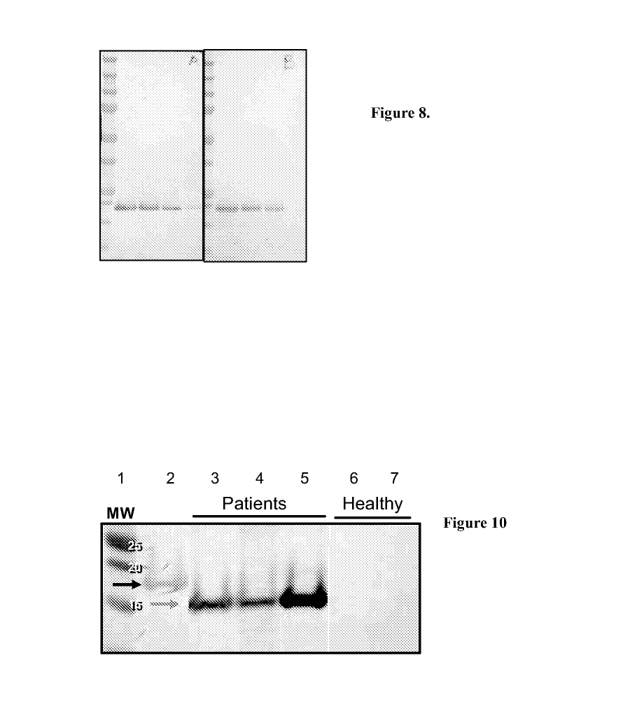

Bacterial cells were collected by centrifugation and lysed by sonication in Buffer 1 (Inclusion Body Wash Buffer—20 mM Tris-HCl, 10 mM EDTA, 1% Triton X-100, pH 7.5). The expressed Reg1 proteins (present in inclusion bodies) were freed of contaminating proteins by two repetitions of Dounce homogenization in Buffer 1 and collection of the pellet. The inclusion body pellet was washed with Buffer 2 (25% isopropyl alcohol), followed by solubilization of the pellet by overnight treatment at 4 degrees C. and 2 hours at room temperature with Buffer 3 (7 M guanidine-HCl, 150 mM reduced glutathione, 0.1 M Tris-HCl, 2 mM EDTA, pH 8.0). Insoluble material was removed by centrifugation and the solubilized protein allowed to slowly refold by dilution in 50 volumes Buffer 4 (0.5 M arginine-HCl, 0.6 mM oxidized glutathione, 50 mM Tris-HCl, pH 8.0) and stirring overnight at 4 degrees C. and the following morning at room temperature. The sample was dialyzed at 4 degrees C. sequentially against Dialysis Buffer I (25 mM Tris-HCl, 25 mM NaCl, 2 mM CaCl₂, pH 7.0) overnight, then Dialysis Buffer II (25 mM MES, 25 mM NaCl, 2 mM CaCl₂, pH 6.0) overnight, and concentrated 10-fold using a tangential flow method with a 10,000 molecular weight cut-off membrane. SDS-polyacrylamide gel electrophoresis (SDS-PAGE) of the proteins in the sample stained with Coomassie Blue showed proteins of the correct size (18 kDalton) and no other visible contaminant proteins. Protein purity of the washed, solubilized and refolded inclusion bodies was estimated to be >98% (FIG. 7). The purified proteins were established to be authentic Reg1A and Reg1B by immunoblot reaction of the proteins with independently derived specific antibodies to Reg1A and Reg1B (FIG. 8). Reg1A and Reg1B purified by this method were used for production of polyclonal antibodies in goats and as protein standards in ELISA assays and immunoblots.

### ELISA Assay for Detection and Quantitation of Recombinant or Fecal Reg1 Proteins

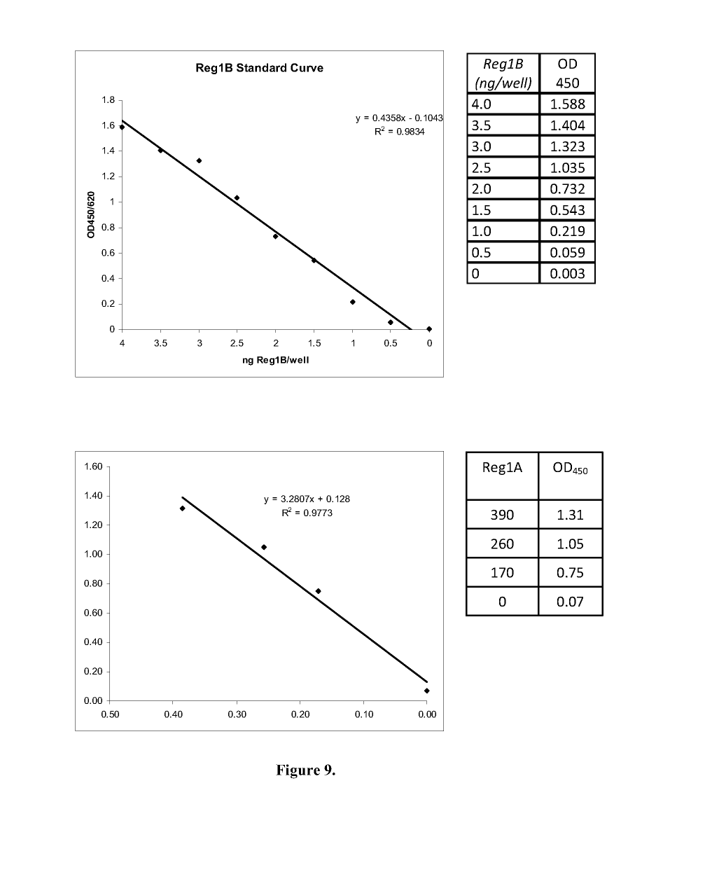

A sandwich ELISA assay was developed for Reg1 proteins, using commercial antibodies. Ninety-six-well plates were coated with 0.25 micrograms of goat anti-Reg1B antibody (R&D Systems, Inc.) or rat monoclonal anti-Reg1A antibody (R&D Systems, Inc.) per well; non-specific binding of spurious proteins was blocked by additional coating of the wells with 5% sucrose, 0.5% BSA, 0.5% Casein in PBS. Known amounts of purified Reg1B protein (between 0.5-4 ng/100 microliters) or 100 microliters of the diluted (1:400) supernatant were added were added to the wells and incubated for 45 minutes at 37° C. The final dilution was obtained by diluting fecal samples 1:5 with PBS followed by centrifugation (10,000 rpm, 5 minutes) then further diluting the supernatant to 1:400 by adding 1.25 microliters of the 1:5 diluted sample into 98.75 microliters of a buffered protein solution (phosphate-buffered saline containing 0.005% gentamicin sulfate, 0.01% thimerosal and 5% horse serum). Wells were washed to remove unbound Reg1 and a second antibody (1:5000 dilution of mouse anti-Reg1B, from R&D Systems, Inc. or a 1:250 dilution of rabbit anti-Reg1A, from BioVendor) was added to each well and incubated another 45 minutes at 37° C. Wells were washed again, to remove unbound antibody, and a horseradish peroxidase (HRP)-coupled antibody specific to goat (for Reg1B) or rabbit (for Reg1A) was added and incubated for a third 45 minute period. Wells were washed; the amount of HRP-coupled antibody that had been captured was detected by addition of substrate (TechLab, Inc) for 10 minutes, stopping the reaction by acidification, and reading the spectrophotometric absorbance at 450 nm of the solution with an ELISA plate reader. Optical density of the solution was correlated with the known concentrations of Reg1 initially added to the wells of the standard curve (FIG. 9). Those skilled in the art will understand that the dilution factor and reagents described may be improved upon or changed and remain within the scope of the invention.

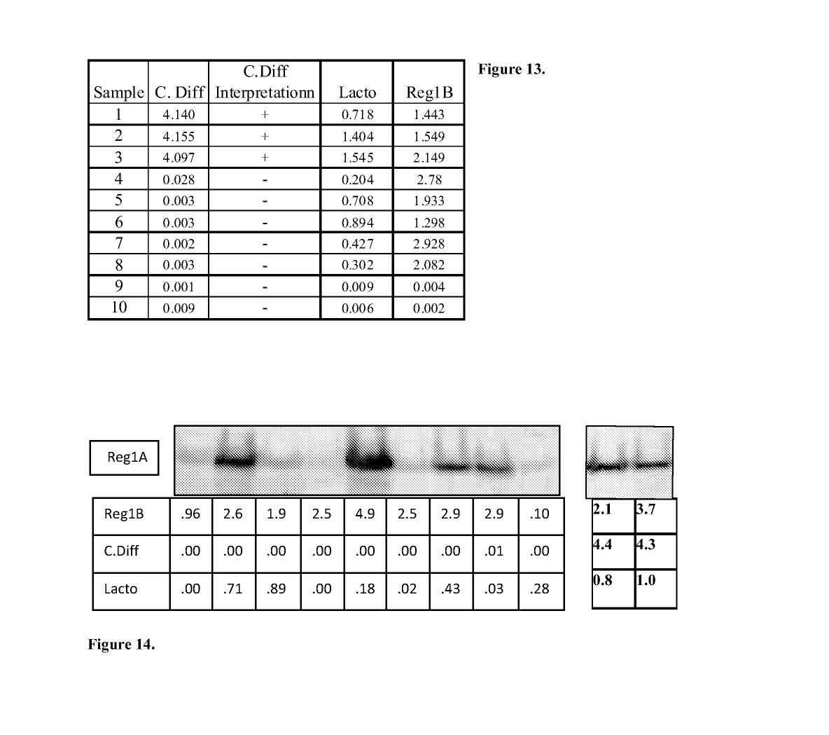

Similar ELISA assays for lactoferrin (IBD-Chek, TechLab, Inc.) or the Clostridium difficile antigen glutamate dehydrogenase (GDH;Chek-60, TechLab, Inc.) were performed on diluted fecal samples according to manufacturer's specifications (FIGS. 13 and 14).

### Immunoblot Detection of Reg1 Proteins in Human Fecal Samples

Human fecal samples were diluted 1:5 with phosphate-buffered saline (PBS) and centrifuged to remove particulates. Samples (5 microliters) were prepared for and separated by SDS-PAGE, and transferred to nitrocellulose membranes. Non-specific protein binding was blocked by incubation of the membranes in 2% w/v bovine serum albumin (BSA) in PBS. Reg1 proteins were detected by incubation of the membranes with either rat monoclonal Reg1A- or goat polyclonal Reg1B-specific antibodies, followed by visualization using HRP-conjugated anti-rat or anti-goat secondary antibodies, respectively (FIG. 10). Recombinant Reg1 proteins were used as positive controls. Fecal Reg1 proteins had undergone proteolytic cleavage (56) in the digestive tract and displayed a smaller apparent molecular weight (˜16 kDalton) than the recombinant proteins.

Immunohistochemistry Showed that REG1A and REG1B Proteins were Increased During the Active Phase of C. difficile Colitis

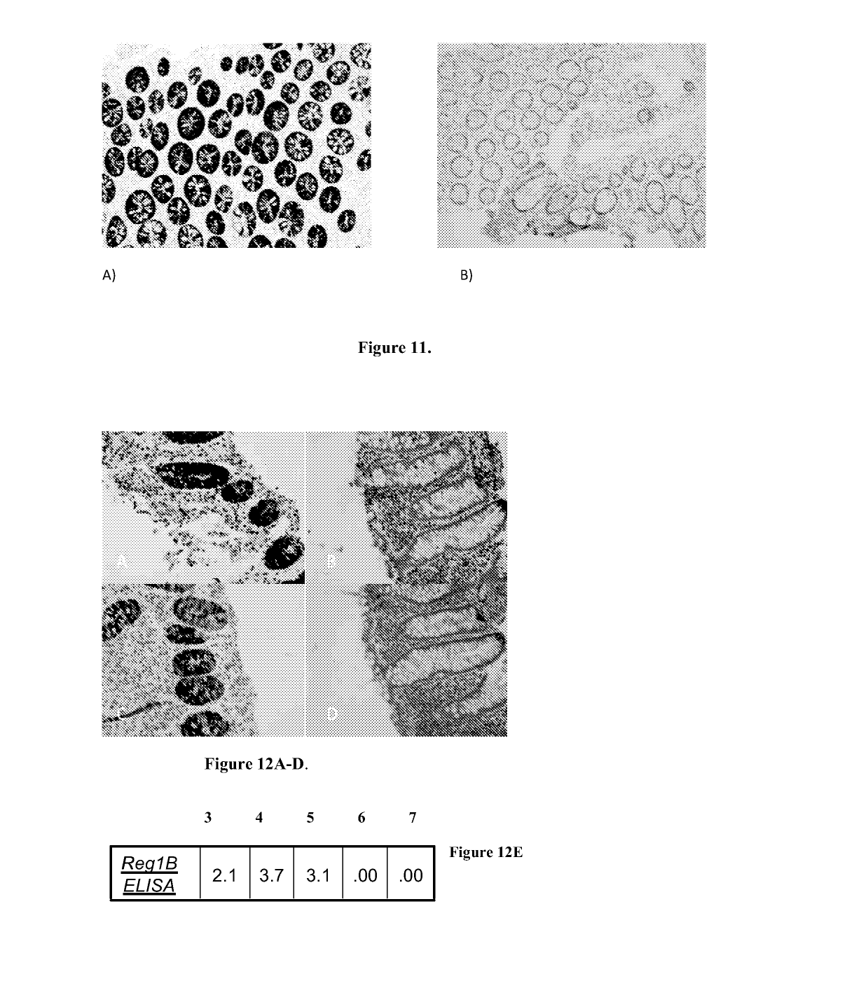

REG1A and REG1B staining was observed in the epithelial cells of the intestinal crypts during active C. difficile colitis with inflammation. Staining was minimal in the absence of inflammation and an unknown, but presumed negative, C. difficile toxin assay (FIG. 11).

Lack of REG1 was Associated with Increased Severity to C. difficile Colitis In Vivo

Mice challenged with VPI 10463 strain of C. difficile at the dose of 10⁵ (A) and 10⁶ (B) cfu were monitored for change in body weight from day 0 to day 6 (presented as mean relative weight±standard error; N=10-11 per group for A, and N=5 per group for B). Reg1 −/− mice showed more weight loss than the WT groups (A. on day 2 the mean relative weight of KO vs WT, P<0.05), and delayed recovery as determined by weight gain at day 6 (B). Pathological changes in the cecum from Reg1 −/− and WT mice challenged with C. difficile were examined. The histological severity of enteritis was graded taking into account the following features: epithelial cell destruction, mononuclear cell infiltration, and edema of the submucosa. The total score of the Reg1 −/− mice were significantly higher than that of the WT controls in groups challenged with both doses (C). Representative histology of antibiotic-treated Reg1 −/− mice exposed to C. difficile showed proliferative ulcerative enteritis with substantial epithelial ulceration and necrosis, associated with submucosal edema and release of inflammatory exudates (D, upper and lower left). In contrast, WT controls exhibited marked mucosal hyperplasia and inflammation with less degree of mucosal ulceration (D, upper and lower right) (FIGS. 1-4).

Reg1A or Reg1B were Strongly Induced in Patients with Diarrhea as Compared to Healthy Donors

Human fecal samples derived from patients with diarrhea or from healthy donors were analyzed for Reg1A and Reg1B content. Immunoblot results for Reg1A showed that healthy donors had very low amounts of Reg1A in stool, while patients had elevated amounts of Reg1A (FIG. 10). The same samples were examined using the Reg1B ELISA assay (FIG. 12). Similar to the Reg1A results, samples from healthy donors showed very low amounts of Reg1B. Patient samples also showed increased amounts of fecal Reg1B.

Reg1A and Reg1B are Elevated in C. difficile-Positive Samples

Additional patient samples that had been tested for the presence or absence of C. difficile antigen were also examined to determine the correlation of Reg1 proteins with infection. In addition, because C. difficile colitis is associated with inflammation, the amount of an inflammatory marker, human neutrophil lactoferrin, which was present in the feces was also determined (FIG. 13). Samples in which large amounts of C. difficile antigen as well as lactoferrin were present, Reg1B was also increased. No samples that were positive for C. difficile antigen and lactoferrin were negative for Reg1B. Similar correlations between C. difficile infection, increased lactoferrin, and Reg1A were also observed (FIG. 14). Reg1A was elevated in C. difficile-positive samples. These results correlate well with the gene microarray and immunohistochemistry results from human colon biopsies from patients with C. difficile colitis and show that increased amounts of Reg1 proteins are present in feces of C. difficile-infected patients.

However, Reg1 proteins can also be elevated in samples in which there is no detectable C. difficile antigen (FIG. 14). In some of these samples, Reg1A and Reg1B are elevated along with lactoferrin. In others, Reg1A or Reg1B are increased when lactoferrin is low. As lactoferrin is produced by invading neutrophils while Reg1A and Reg1B are secreted by the epithelial cells that line the gastrointestinal tract, Reg1A and Reg1B respond to and report different events than lactoferrin.

### BIBLIOGRAPHY

1. Bartoli, C. et al. FEBS Lett. 327: 289-293, 1993.
2. Miyashita, H. et al. FEBS Lett. 377: 429-433, 1995.
3. Moriizumi, S. et al. Biochim. Biophys. Acta 1217: 199-202, 1994.
4. Shinozaki S.et al. Gut. 2001;48:623-629.
5. Steele I A, et al. Am J Physiol Gastrointest Liver Physiol 2007, 293:347-54.
6. Fukui H, et al. Lab Invest 2003;83:1777-1786.
7. Lawrance I C, Fiocchi C, Charkravarti S. Hum Mol Genet. 2001;10:445-56.
8. Dieckgraefe B K, et al. Physiol Genomics. 2000;4:1-11.
9. Geng J, et al. European Journal of Surgical Oncology (EJSO), Vol. 35(8) (852-857).
10. Sekikawa A, et al. Gut. 2005;54:1437-1444.
11. Cavard C, et al. Oncogene 2006, 25:599-6089.
12. Sekikawa A, et al. Carcinogenesis. 2007;29:76-83.
13. Kazumori H, et al. J Lab Clin Med 2002, 139:101-8.
14. Brandl K, et al. Nature. 2008;45:804-808.
15. Brandl K, et al. JEM. 2007;204:1891-1900.
16. Cash H, Whitham C, Behrendt C, Hooper L. Science. 2006;313:1126-1130.
17. Mondal D, et al. Trans R Soc Trop Med Hyg. 2006;100:1032-8.
18. Fotedar R, et a l. Clin Microbiol Rev. 2007;20(3):511-32.
19. Stanley S L, Jr. Lancet. 2003;361:1025-34.
20. Blazquez S, et al. Int J Parasitol. 2007;37(3-4):425-33.
21. Sanchez D, et al. Biochem Biophys Res Commun. 2001;284(3):729-37.
22. Watanabe T, et al. J Biol Chem. 1990;265(13):7432-9.
23. Senegas-Balas F O, et al. J Histochem Cytochem. 1991;39(7):915-9.
24. Dieckgraefe B K, et al. J Investig Med. 2002;50(6):421-34.
25. Sekikawa A, et al. Gastroenterology. 2005;128(3):642-53.
26. Sekikawa A, et al. 2005;54(10):1437-44.
27. Unno M, et al. Diabetes. 2002;51(suppl 3):5478-83.
28. Sekikawa A, et al. Lab Invest. 2010;90(3):496-505.
29. Kawanami C, et al. J Gastroenterol. 1997;32(1):12-8.
30. Fukuhara H, et al. Lab Invest. 2010;90(4):556-65.
31. Gross D J, et al. Endocrinology. 1998;139(5):2369-74.
32. Fukui H, et al. Gastroenterology. 1998;115:1483-93.
33. Shinozaki S, et al. Gut. 2001;48:623-9.
34. Sekikawa A, et al. Carcinogenesis. 2008;29(1):76-83.
35. Huston C D, et al. Cell Microbiol. 2000;2:617.
36. Ryckman C, et al. J Immunol. 2003;170:3233-42.
37. Takito J, Al-Awqati Q. J Cell Biol. 2004;166(7):1093-102.
38. Madsen J, et al. Eur J Immunol. 2003;33:2327-36.
39. Grover P K, et al. Cancer Metastasis Rev. 2010;29(4):761-75.
40. Zhang X, Huang Q, Yang Z, Li Y, Li C Y. Cancer Res. 2004;64:2474-81.
41. Dieckgraefe B K, et al. Physiol Genomics. 2000;4:1-11.
42. Renner M, et al. Gastroenterology. 2007;133:1499-509.
43. Te Velde A A, et al. Eur J Gastroenterol Hepatol. 2008;20(6):555-60.
44. Zhang Z, Stanley S L, Jr. Cell Microbiol. 2004;6(6):535-54.
45. Borenshtein D, et al. Genome Biol. 2008;9(8):R122.
46. De Reggi M, Gharib B (2002) Curr. Protein Pept. Sci. 2 (1): 19-42.
47. Itoh T., et al. (1990) FEBS Lett. 272 (1-2): 85-8.
48. de la Monte S M, Ozturk M, Wands J R (1990) J. Clin. Invest. 86 (3):1004-13.
49. Watanabe T, et al. (1990). J. Biol. Chem. 265 (13): 7432-9).
50. Karlin and Altschul (1990) Proc. Natl. Acad. Sci. USA 87:2264-2268.
51. Karlin and Altschul (1993) Proc. Natl. Acad. Sci. USA 90:5873-5877.
52. Altschul, et al. (1990) J. Mol. Biol. 215:403-410.
53. Altschul et al. (1997) Nucleic Acids Res. 25:3389-3402.
54. Devereux et al., 1984 Nucl. Acids Res. 12:387.
55. Altschul et al., 1990 Proc. Natl. Acad. Sci. USA. 1990 87:14:5509-13.
56. Rouimi, P., et al. 1987. FEBS Letters 216:195-199.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method for diagnosing infectious or inflammatory diarrhea comprising detecting the presence of Reg1 protein or mRNA in a fecal sample or intestinal biopsy from a subject, wherein the presence of Reg1 protein or mRNA in the fecal sample or intestinal biopsy indicates that the subject has infectious and/or inflammatory diarrhea.

2. The method of claim 1, wherein Reg1 protein is detected in the fecal sample.

3. The method of claim 1, wherein Reg1 protein or mRNA is detected in the intestinal biopsy.

4. A method for diagnosing infectious or inflammatory diarrhea comprising obtaining a fecal sample from a subject, determining the sample contains Reg1 protein, and diagnosing the subject with infectious and/or inflammatory diarrhea.

5. A method for diagnosing infectious or inflammatory diarrhea comprising obtaining an intestinal biopsy from a subject, determining the biopsy contains Reg1 mRNA or protein, and diagnosing the subject with infectious and/or inflammatory diarrhea.

6. A method for determining if treatment of infectious or inflammatory diarrhea is efficacious comprising detecting the presence of Reg1 protein or mRNA in a fecal sample or intestinal biopsy from a subject, wherein the presence of a reduced amount Reg1 protein or mRNA in the fecal sample or intestinal biopsy as compared to a sample from the subject taken earlier indicates that the treatment is effective.

7. The method of claim 6, wherein Reg1 protein is detected in the fecal sample.

8. The method of claim 6, wherein Reg1 protein or mRNA is detected in the intestinal biopsy.

9. The method of claim 1 any one of claims 1 8, wherein Reg1 protein is detected by an immunological method which comprises contacting said sample with at least one reagent containing at least one antibody which binds to a Reg1 protein.

10. The method of claim 9, wherein the antibody is detected using at least one of enzyme-linked immunoassay, western blot, lateral flow membrane test, latex agglutination, or other forms of immunochromatography or immunoassay utilizing at least one antibody.

11. The method of claim 1, wherein Reg1 protein is detected using an ELISA.

12. A method to treat infectious and/or inflammatory diarrhea comprising administering to a subject in need thereof an effective amount of at least one Reg1 protein, or an active homolog, variant or fragment thereof.

13. Prophylaxis treatment of infectious and/or inflammatory diarrhea comprising administering to a subject an effective amount of at least one Reg1 protein, or an active homolog, variant or fragment thereof.

14. The method of claim 1, wherein the Reg1 is Reg1A, Reg1B or a combination thereof.

15. The method of claim 1, wherein the subject is human.

16. The method of claim 1, wherein the diarrhea is caused by a bacterium or parasite.

17. The method of claim 16, wherein the bacterium or parasite is a Campylobacter jejuni, Salmonella species, Shigella species, Escherichia coli (including enterohemorrhagic, enterotoxigenic, enteroaggregative E. coli), Entamoeba histolytica, Clostridium difficile, Cryptosporidium or a combination thereof.

18. The method of claim 1, wherein the diarrhea is caused by Clostridium difficile.

19. The method of claim 1, wherein the diarrhea is caused by Entamoeba histolytica.

20. The method of claim 1, wherein the diarrhea is due to Crohn's Disease (CD), ulcerative colitis (UC) or other chronic noninfectious inflammatory diarrheas.

21. The method of claim 1 further comprising informing a health care provider.

22. The method of claim 1 further comprising treating the subject for infectious or inflammatory diarrhea following diagnosis of infectious or inflammatory diarrhea.


SEQUENCE LISTING (4 sequences)

SEQ ID NO: 1 - DNA, 821 bp, Homo sapiens
gatataaagctcctacagctacctggcctgagaagccaactcagactcagccaacagagattgttgatttgcctcttaagcaagagattcattgcagctcagcatggctcagaccagctcatacttcatgctgatctcctgcctgatgtttctgtctcagagccaaggccaagaggcccagacagagttgccccaggcccggatcagctgcccagaaggcaccaatgcctatcgctcctactgctactactttaatgaagaccgtgagacctgggttgatgcagatctctattgccagaacatgaattcgggcaacctggtgtctgtgctcacccaggccgagggtgcctttgtggcctcactgattaaggagagtggcactgatgacttcaatgtctggattggcctccatgaccccaaaaagaaccgccgctggcactggagcagtgggtccctggtctcctacaagtcctggggcattggagccccaagcagtgttaatcctggctactgtgtgagcctgacctcaagcacaggattccagaaatggaaggatgtgccttgtgaagacaagttctcctttgtctgcaagttcaaaaactagaggcaactggaaaatacatgtctagaactgatccagcaattacaacggagtcaaaaattaaaccggaccatctctccaactcaactcaacctggacactctcttctctgctgagtttgccttgttaatcttcaatagttttacctaccccagtctttggaaccctaaataataaaaataaacatgtttccactattgtgctgtcaaaaaaaaaaaaa

SEQ ID NO: 2 - protein, 166 aa, Homo sapiens
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 3 - DNA, 812 bp, Homo sapiens
gatataaagctcctactctgtctgacctgacaagccacctcaagtggacaaggcacttaccaacagagattgctgatttgctccttaagcaagagattcactgccgctaagcatggctcagaccaactcgttcttcatgctgatctcctccctgatgttcctgtctctgagccaaggccaggagtcccagacagagctgcctaatccccgaatcagctgcccagaaggcaccaatgcctatcgctcctactgctactactttaatgaagaccctgagacctgggttgatgcagatctctattgccagaacatgaattcaggcaacctggtgtctgtgctcacccaggcggagggtgccttcgtggcctcactgattaaggagagtagcactgatgacagcaatgtctggattggcctccatgacccaaaaaagaaccgccgctggcactggagtagtgggtccctggtctcctacaagtcctgggacactggatccccgagcagtgctaatgctggctactgtgcaagcctgacttcatgctcaggattcaagaaatggaaggatgaatcttgtgagaagaagttctcctttgtttgcaagttcaaaaactagaggaagctgaaaaatggatgtctagaactggtcctgcaattactatgaagtcaaaaattaaactagactatgtctccaactcagttcagaccatctcctccctaatgagtttgcatcgctgatcttcagtaccttcacctgtctcagtctctagagccctgaaaaataaaaacaaacttatttttatccagtgaaaaaa

SEQ ID NO: 4 - protein, 166 aa, Homo sapiens
MAQTNSFFMLISSLMFLSLSQGQESQTELPNPRISCPEGTNAYRSYCYYFNEDPETWVDADLYCQNMNSGNLVSVLTQAEGAFVASLIKESSTDDSNVWIGLHDPKKNRRWHWSSGSLVSYKSWDTGSPSSANAGYCASLTSCSGFKKWKDESCEKKFSFVCKFKN